(19) United States
(12) Patent Application Publication
(10) Pub. No.: US 2021/0121538 A1
(43) Pub. Date: Apr. 29, 2021

(54) Interactional Biosystem

(71) Applicant: Nobelbiotix Research Institute Limited, Hong Kong (HK)

(72) Inventor: Winston Kuang Hui Man A Hing, Hong Kong (HK)

(21) Appl. No.: 17/072,005

(22) Filed: Oct. 15, 2020

Related U.S. Application Data
(60) Provisional application No. 62/925,205, filed on Oct. 23, 2019.
(60) Provisional application No. 62/945,854, filed on Dec. 9, 2019.

(51) Int. Cl. A61K 38/44; A61K 47/46; A61K 47/36; A61K 47/02; A61K 47/06; A61K 47/20; A61K 47/32; A61K 47/44; A61K 47/28; A61K 47/18; A61K 45/06; A61K 31/375; A61K 31/194; A61K 31/122; A61K 31/015; A61K 31/355; A61K 31/353; A61K 31/593; A61K 31/352; A61K 31/336; A61K 31/4415; A61K 31/525; A61K 31/714; A61K 31/455; A61K 31/198; A61K 31/131; A61K 31/7076; A61K 31/664; A61K 31/10; A61K 38/05; A61K 38/06; A61K 31/7032; A61K 31/7084; A61K 35/76; A61K 35/748; A61K 36/05; A61K 36/06; A61K 35/74

(52) U.S. Cl. CPC A61K 38/446; A61K 35/74; A61K 47/36; A61K 47/02; A61K 47/06; A61K 47/20; A61K 47/32; A61K 47/44; A61K 47/28; A61K 47/18; A61K 45/06; A61K 31/375; A61K 31/194; A61K 31/122; A61K 31/015; A61K 31/355; A61K 31/353; A61K 31/593; A61K 31/352; A61K 31/336; A61K 31/4415; A61K 31/525; A61K 31/714; A61K 31/455; A61K 31/198; A61K 31/131; A61K 31/7076; A61K 31/664; A61K 31/10; A61K 38/44; A61K 38/05; A61K 38/063; A61K 31/7032; A61K 31/7084; A61K 35/76; A61K 35/748; A61K 36/05; A61K 36/06; A61K 47/46

(57) ABSTRACT

A micro-environment bioreaction aggregate (“MEBA”) is formed by combining a non-digestible carrier system with redox potential (“Eₕ”) regulating compounds. The bioreaction aggregate is introduced into a microbial environment, such as soil, hydroponic systems, or gastrointestinal tracts of animals or humans. Said MEBA regulates the Eₕ of the microbial microhabitat and its surroundings, thereby providing numerous benefits towards the prevention and treatment of diseases and dysbiosis.

### FIELD

The present invention relates to a method for preparing, and composition of, a micro-environment bioreaction aggregate (“MEBA”) to influence microbial biomass activity.

The obtained composition may be used for the prophylaxis or treatment of diseases in plants, animals, and humans, particularly related to a disturbance of the oxidation-reduction conditions and the redox potential (“Eₕ”) of the microbial microhabitat and its surroundings.

### BACKGROUND

### Dysbiosis in Agriculture and Soil

The continuing decline in soil health is a global issue. Recent studies demonstrate that the planet lost 33% of its arable land due to erosion or pollution over the last forty years. This can have disastrous consequences as the global demand for food exponentially increases. This trend could become irreversible unless new innovative solutions to tackle this problem are found.

Many modern agriculture practices deplete soil microbes. The resulting imbalance in microorganisms, or dysbiosis, increases disease and reduces nutrient uptake. The microbial biomass and diversity of soil declined significantly with the rise of intensive agriculture. This negatively impacts yield, increases spread of disease, and reduces farm sustainability.

Certain agrochemicals have been shown to have a strong negative impact on soil enzyme activity (Moeskops 2010; Ratnadass 2012). Other studies demonstrate that the frequent use of fungicides, herbicides and insecticides caused a decrease in bacterial and fungal-feeding symbiotic nematodes and an increase in plant-parasitic nematodes (Geense et al 2015; Houlden 2003; Imfeld 2012). Additional examples are annual broadacre crops, where microbes soon die once the crops are harvested, and the roots are no longer present. Cultivation and fumigation of soil, common during vegetable production, destroys roots and beneficial microbiota. In fruit orchards and vineyards, spraying against weeds with herbicides causes plant roots and beneficial microbes to die.

Biodiversity of soil microbiota is crucial for both nutrient uptake and disease suppression. Soil microbes interact with the roots of plants in different ways. They can develop symbiotic relationships with plant roots to protect the plant from disease. They release nutrients from organic matter to stimulate plant growth, fix atmospheric nitrogen, and improve soil structure. Additionally, microbiota also help degrade pesticides and toxic substances. The incidence and severity of root disease is an indirect assessment of soil health (Abawi 2000).

### Dysbiosis in Aquaculture and Animals

The symbiotic relationship between the microbiota and animals is an increasingly well-researched field, especially within the gastrointestinal tract. The health of this tract is crucial for the general health and well-being of animals.

According to the United Nations Food and Agricultural Organization (“FAO”), world food fish aquaculture production expanded at an average annual rate of 6.2% in the period 2000-2012 (FAO, 2014). Intensive aquaculture has increased the severity and frequency of diseases in farmed fish and shrimp. Traditionally, antibiotics have been widely applied to prevent and treat diseases in aquacultures. However, antibiotic abuse leads to transfer of resistant genes among pathogens, and it has raised concerns regarding environmental pollution and consumer safety.

For example, Tilapia, one of the larger fish in aquaculture is normally a hardy species originating from lakes in East Africa. But in aquaculture, the fish become more sensitive to pathogens than in its natural habitat. When treated with a regimen of probiotics comprised of lactobacillus strains, the fish stayed healthy. However, as soon as the regimen was completed, the fish developed gut microbiota dysbiosis similar to that caused by antibiotics, leading to a diminished resistance against infections. Furthermore, the lactobacilli could not be detected in the gut after the end of the use of the probiotic. Future aquaculture of Tilapia depends on an effective remedy to this growing concern.

Similarly, intensive use of antibiotics as growth promoters in farmed land animals, like cattle and pigs, is practiced in numerous countries including the United States. While many are phasing out the use of antibiotics, probiotics began to replace them. However, probiotics can produce bacteriocins with similar effects to antibiotics. Additionally, cessation of these probiotics can often lead to antibiotic associated diarrheas.

In addition to the effects of antibiotic or probiotic regimens, livestock's feed intake and efficient absorption of nutrients is determined by the health of their gastrointestinal tract in general. This is particularly true in poultry farming, where intensive selection for fast weight gain and low feed conversion ratio has developed breeds with an extremely high feed intake. Certain feed ingredients, and an excessive amount of feed, can put stress on the digestive system. Even in the absence of any specific pathogens, this can negatively influence the health status of poultry and impair gastrointestinal function leading to malabsorption or diarrhea.

Animals kept as pets (e.g., dogs and cats) and for leisure (e.g., horses) have similar problems. Pet cats and dogs live in close proximity to humans and have similar environmental exposures. They are also afflicted by many of the same complex diseases as humans, including metabolic syndrome, obesity, diabetes, inflammatory bowel diseases, and cancers, all of which may be influenced by diet and microbiota. A horse's cecum is developed for fermentation of fibrous digestive products, but with the wrong type of feed, can still develop metabolic syndrome type problems.

Different approaches have been discussed to treat dysbiosis in animals. As mentioned above there are ongoing attempts to add probiotics to the feed in aquaculture and farming. This may cause new problems, as exemplified with Tilapia. Upon cessation of the probiotic treatment, antibiotic like effects deplete the biodiversity of the resident and may cause gastrointestinal problems.

### Dysbiosis in Humans

Microbiome research has revealed that microbiota, capable of both beneficial and pathogenic activity, exist in nearly every portion of the human body, including blood and tissue. Lactic acid bacteria, for example, protect their ecological niches by producing lactic acid, antibacterial peptides (bacteriocins), and H₂O₂. This counteracts vaginal infections. If the lactic acid bacteria population declines, possibly from the use of antibiotics or oral contraception pills, their protective function inevitably declines as well. In response, epithelial tissues are prone to staphylococcal infection and overgrowth of Candida albicans.

Candidiasis is the most common vaginal infection, affecting about 50-72% of women, with 40-50% having recurrent episodes. If untreated, vaginal candidiasis can lead to chorioamnionitis, with subsequent abortion or prematurity in pregnant women, congenital infection of the neonate, or pelvic inflammatory disease resulting in infertility. Despite treatment with antifungals, relapses are very common. The same is true with lactobacillus replacement therapy (e.g., by vagitories).

The gastrointestinal tract of a human is even more closely associated with microbiota. The average human intestine is a habitat for about 300-1000 different species of bacteria. The number of microbial cells within the gut lumen is approximately equal to the number of eukaryotic cells in the human body. As a result, intestinal microbiota is involved not only in regulating digestion, producing essential vitamins and minerals, but also in maintaining host physiology, steering key immune functions, and assisting in maintaining the gut barrier. Alterations in the microbiota equilibrium are the underlying cause of gastrointestinal conditions and many other metabolic and auto-immune diseases (obesity, allergies, autism, etc.) Increasing scientific evidence of the importance of the interaction between a balanced gut microbiota and its host has been mounting in recent years.

Infections, antibiotic treatment, and an unphysiological diet (e.g., a high caloric diet with high fructose contents) are examples of external factors that may disrupt the ecological balance of the intestinal microflora. Dysbiosis may result in a reduced diversity of the gut microbiota, leading to a disruption of the anaerobicity in the colon, which in turn can lead to an increased aerobic glycolysis within human cells, toxin production (e.g., methylglyoxal), and oxidative stress.

The culprit of dysbiosis is the change in the micro-environment of the gastrointestinal tract. The gut is a three-dimensional tube with the mucosa surface area of different segments largely increased by folds, villi, and crypts. The Eₕ of the gastrointestinal tract varies, being highest in the oral cavity and lowest in the cecum and colon. In the gastrointestinal tract, there is an intense communication and interaction on a molecular level between individual microorganisms like bacteria, archaea, fungi, and viruses, enterocytes, the nervous system, and the immune system. The biochemical processes are influenced by the Eₕ status, and unphysiological changes in Eₕ may promote the development of disease.

Dysbiosis can disrupt the metabolic balance and allow certain microbes that operate at higher Eₕ to establish themselves where they normally could not and should not. For example, if anaerobicity of the colon is disrupted, fragments of microbial biofilm plaques containing Fusobacteria can be dislodged from the oral cavity and survive passage through the gastrointestinal tract and colon. Because of dysbiosis, the Eₕ is not low enough to prevent the Fusobacteria establishing themselves in the colon, possibly contributing to colon cancer. Fusobacterium nucleatum also has the capacity to create agglomerations with other more oxygen sensitive bacteria and allow them to work in oxidized environments. The change in local Eₕ shifts the microbiota to release more metabolites in a more oxidized form, leading to further disruption of the ecological balance.

Alterations of intestinal microbiota may be caused by consumption of antibiotics or pathogen infections. Clostridium difficile, an anaerobic, gram-positive bacterium, is a major cause of antibiotic-associated diarrhea, and challenges healthcare infection control measures by producing highly infectious and resistant spores. Antibiotic treatment, advanced age, and hospitalization are major risk factors for C. difficile colonization, which could cause an asymptomatic carriage, severe diarrhea, pseudomembranous colitis or death. Current first line treatments for C. difficile disease are vancomycin or metronidazole, although 20-35% of cases relapse following the cessation of antibiotic therapy. Recurrent C. difficile disease is associated with a pathological imbalance of the resident intestinal microbial community. As a result, therapies that restore a healthy microbiota are regarded as good alternatives.

Several approaches, have been tried to fight the problem of dysbiosis, including prebiotics, probiotics, and preparations with fecal bacteria. Each of these have their own problems, and they do not address the key problem, the change in Eₕ.

Prebiotics are fibers that can stimulate the growth of certain “friendly bacteria” in the colon. If the Eₕ conditions are not right, and there is a higher oxidative state in the colon, the result can be discomfort, gas production, and diarrhea. This is the case with many Irritable Bowel Syndrome patients, whose symptoms worsen after receiving prebiotics.

Probiotics are bacteria that are normally friendly, and may be diminished in certain conditions like Irritable Bowel Syndrome. Lactobacillus rhamnosus is one example of a common probiotic. However, Lactobacillus rhamnosus produces a bacteriocin that inhibits other er, e Irritable Bowel Syndrome. n, the result can be discomfort, gas production, and diarrhea. This is the case with many Irritable Bowel Syndrome patients, whose symptoms worsen after receivinge combat their closest relatives, and also other bacterial species in the healthy gut microbiota. This can result in an aggravation of the dysbiosis.

Fecal bacteriotherapy, also known as fecal matter transplantation (“FMT”), is the administration of homogenized feces from a healthy donor. It is an alternative therapy for recurrent C. difficile disease in humans (Nood et al. 2013).

FMT is a known method, but not without risk. Until now fecal material from donors of close relatives has been used. A main problem with this procedure is the risk of transferring bacterial genes conferring antimicrobial resistance to certain antibiotics through horizontal gene transfer. The transfer of such genes may result in serious consequences for the patient, as it may compromise the efficacy of future antibiotic treatment. Another issue with FMT is the risk of transmitting potentially contagious agents present in the donor's feces. This requires careful, time-consuming, and expensive screening of the feces and the donor.

A new treatment of plant animal and human dysbiosis is needed, that addresses the Eₕ of the microbial environment directly, rather than introducing antibiotics or probiotics in the form of foreign microbiota.

### REFERENCES

Moeskops, et al., Soil microbial communities and activities under intensive organic and conventional vegetable farming in West Java, Indonesia, Applied Soil Ecology 45 (2010) 112-120.

Ratnadass, et al., Plant species diversity for sustainable management of crop pests and diseases in agroecosystmes: a review, Agronomy for Sustainable Development (2012) 273-303.

Geense, et al., Can Changes in Soil Properties in Organic Banana Production Suppress Fusarium Wilt?, Natural Resources (2015) 181-195.

Houlden, et al., Influence of plant developmental stage on microbial community structure and activity in the rhizosphere of three field crops, FEMS Microbiology Ecology (2008) 193-201.

Imfeld, et al., Measuring the effects of pesticides on bacterial communities in soil: A critical review, European Journal of Soil Biology (2012) 22-30.

Abawi, et al., Impact of soil health management practices on soilborne pathogens, nematode and root diseases of vegetable crops, Applied Soil Ecology (2000) 37-47.

Food and Agriculture Organization of the United Nations, The State of World Fisheries and Aquaculture (2014) 6.

Nood, et al., Duodenal Infusion of Donor Feces for Recurrent Clostridium difficile, The New England Journal of Medicine (2013) 407-415.

Bong-Soo Kim, et al., In Vitro Culture Conditions for Maintaining a Complex Population of Human Gastrointestinal Tract Microbiota, Journal of Biomedicine and Biotechnology (2011) 1-10.

### SUMMARY

The invention described herein addresses the Eₕ of the microbial environment that contributes to dysbiosis in microbial microhabitats.

Disclosed is a method for creating a composition for introduction into a microbial environment, for the purpose of preventing or treating dysbiosis. The composition is comprised of a Carrier System and Compound Matrix.

The Carrier System is an inert substance, that provides a structural backbone for the Compound Matrix in the targeted environment. The Carrier System may also act as an electron acceptor and/or conductor. Together the Carrier System and Compound Matrix form a MEBA which provides a three-dimensional porous structure for the safe growth of beneficial microbiota.

The Compound Matrix is a single substance, or combination of substances, with the ability to regulate the Eₕ of a microbial environment.

These two components, the Carrier System and the Compound Matrix, combine to form a MEBA. The MEBA is a porous structure with a regulated redox potential (Eₕ) environment. It provides a habitat for microorganisms, including but not limited to bacteria, affording them a place to thrive, in a refuge from a hostile environment with disadvantageous redox potential levels. The porous structure constitutes micro-ecological niches for the microorganisms, facilitating the interaction between the environment outside the MEBA and inside the MEBA, including the exchange of signalling substances, biochemical metabolites, and gases. The microorganisms in the MEBA can also interact with plant, animal, or human cells in the surrounding environment. The MEBA structure can be adapted to remain intact, or to dissolve at a certain time, releasing the microbiota and its milieu established in the MEBA in other parts of the gastrointestinal system or soil. Other substances may be added to the MEBA, including Supernatants, providing metabolic factors to aid the colonization and growth of beneficial microbiota, and Excipients to aid administration to the target environment.

### DETAILED DESCRIPTION

The present invention provides a composition, and method for creating said composition, that addresses the Eₕ of a microbial environment in order to prevent and reverse dysbiosis.

### Solving Redox Potential in Microbial Environments

The habitat of microorganisms in various ecosystems is regulated by the external environment and its chemical composition in terms of nutrients, minerals, oligoelements, and their influence on pH, but also on the Eₕ.

At its most basic, Eₕ, measured in mV, of an environment refers to its tendency to acquire or lose electrons to an electrode. Oxidation is the loss of electrons or an increase in the oxidation state while reduction is the gain of electrons or a decrease in the oxidation state.

The environmental contributions to the Eₕ can determine where certain microbes live. For example, aerobic and anaerobic microbes thrive in vastly different conditions. The higher the Eₕ of a given bacterial environment, the higher the oxygen concentration, which is essential for aerobic bacterial growth. Lowering the Eₕ in experimental conditions, or in naturally lower Eₕ environments, provides the necessary environment for anerobic microbes. If the Eₕ conditions around a certain microbe changes, the microbe may no longer thrive or may die. A disruption of the Eₕ within a particular environment can lead to disruption of its ecological balance and dysbiosis. This dysbiosis can have disastrous consequences for plants living in natural soil, artificial soil, or hydroponic culture.

Adding microbes to the damaged ecosystem is not enough to restore optimal soil conditions. We have found it is crucial to maintain the right Eₕ homeostasis. Low organic matter content and oxidized soil conditions lead to low fertilizer efficiency in the short term, because plants must release a large share of their photosynthetic production as root exudates to adjust the Eₕ in the rhizosphere, ensuring cell homeostasis. It is therefore essential to restore the Eₕ balance first, allowing the bacteria already present or added to develop, and safely interact with the rhizosphere of plants and the surrounding environment.

The present invention solves this problem to normalize the Eₕ in environments where the microbiota has been disturbed or is in an imbalance. The microbes themselves can influence the Eₕ of their environment. However, in soils that have been leeched out, overfertilized, or exposed to microbiocidal substances like certain pesticides, glyphosate, and antibiotics from farm or aquaculture waste, the number of microbes and their biodiversity are reduced. Those that remain can no longer contribute to balancing the redox system in the soil.

The invention unexpectedly also solves the problem related to a disturbed microbiota of the gastrointestinal tracts of animals. In industrialized animal husbandry, pigs, chickens, and other livestock are reared by the thousands in cramped conditions. This puts an enormous stress on their immune systems, and many animals are given high doses of antibiotics and other antimicrobials, which may exacerbate dysbiosis in animals' gastrointestinal tracts.

The present invention provides a solution to this problem by addressing the Eₕ of the gastrointestinal tract. As described in soil above, the Eₕ is adjusted in order to create a favorable environment for microbiota to live.

The invention can be used to reestablish the Eₕ, in damaged soils by allowing a healthy microbiota to survive in the rhizosphere, and in animals and humans by allowing a healthy microbiota to form in affected gastrointestinal tracts.

### Carrier System

This invention provides a novel composition comprising a Carrier System and a Compound Matrix. When combined, these two components form a MEBA. The MEBA is added to an affected microbial environment in order to adjust the Eₕ and create conditions conducive to beneficial microbiota and enhanced biodiversity.

The Carrier System is a single substance or combination of substances.

The Carrier System substances are inert and not readily digestible. The Carrier System may include Minerals, Aerogels, Metallic Ions, Carbon Allotropes, Charcoal, Chitin-Glucan Complexes, Quinones, Resins, Glycosaminoglycans, Polysaccharides, Latex, Waxes, and Lipids, in addition to other substances.

Example Mineral based substances include Pumice, Loess, Clay, Diatomite, Zeolite, Clinoptilolite, Bentonite, Kaolinite, Silica, Quartz, and Silt.

Example Metallic ions include, Fe2+, Fe3+, Mn2+, Mn4+ or salts or substances rich in such ions.

Example Carbon Allotropes include Graphene, and Graphene aerogels.

Example Charcoals include Wood Charcoal, Sugar Charcoal, and Active Carbon.

Example Chitin-Glucan Complexes include Chitin, Glucans, and Chitin-Glucan Complexes.

Example Quinones include anthraquinone-2,6-disulfonic acid, anthraquinone-2-sulfonic acid, and compounds rich in quinones.

Example Resins includes Rosin and its glycerol, sorbitol, and mannitol esters.

Example Glycosaminoglycans include Chondroitin, Chondroitin Sulphate, and Hyaluronic Acid.

Example Polysaccharides include Alginate, Carrageenan, Agar, Guar Gum, Arabic Gum, Starches, and Pectin.

Example Waxes and Lipids include Bee's wax, Carnauba wax, Candelilla wax, Spermaceti, Ceramide, Sphingolipids, Bile acid, Cholesterol, Chenodeoxycholic acid, Ursodeoxycholic acid, Hyodeoxycholic acid, Squalane, and Squalene.

The Carrier System has multiple functions. One is to provide a three-dimensional porous network and structural backbone for the Compound Matrix. Another is to provide a safe location for microbiota to propagate and interact with the surrounding environment. Depending on its composition the Carrier System may also function to aid the transfer of electrons.

The Carrier system may be adapted by various formulations and techniques to optimize its use in soil, an aquatic environment, or a gastrointestinal tract.

### Compound Matrix

The Compound Matrix is a single substance or combination of substances with the ability to regulate the Eₕ of a microbial environment. The Compound Matrix may include Antioxidants, Vitamins, Substances with thiol-groups, Amino Acids, Peptides, Polyamines, Enzymes, Nucleotide derivatives, Quorum Sensing Substances, Phytohormones, and Fractionated Nonviable Microbes including viruses, in addition to other substances.

Example Antioxidants include Ascorbic acid, Citric acid, Astaxanthin, Carotenes, Reduced Metal ions, Polyphenols, Flavonoids, Fucoxanthin, Phycocyanin, Xanthines, Catechins, Ellagic acid, Chlorophyll, and Cu-Chlorophyll.

Example Vitamins include A, B1, B2, B3, B6, B12, C, E, D, and K1.

Example substances with thiol groups include Cysteine, Glutathione, Thioredoxin, Methionine, Coenzyme A, Coenzyme B, Coenzyme M, Homocysteine, Lipoic acid, Mycothiol, Bacillithiol, γ-Glu-Cys, Trypanothione, Ergothioneine, Glutathione amide, Mono-galactosyl diglyceride and other galactolipids.

Example Amino Acids and Peptides include reduced forms of Serine, anti-microbial peptides preferably 10 to 50 residues long, and other bioactive oligopeptides.

Example Polyamines include Spermidine, Spermine, Putrescine, Cadaverine, and Agmatine.

Example Enzymes include Catalase, Superoxide dismutase, and Quinone reductase.

Example Nucleotide derivatives include Nicotinamide Adenine Dinucleotide (NAD), Nicotinamide Adenine Dinucleotide Phosphate (NADP), and Flavin Adenine Dinucleotide (FAD).

Example Quorum Sensing Substances include Indolic compounds, Indole, Indole-3 acetic acid, Indole-3 butyric acid, and Indole-3 propionic acid.

Example Phytohormones include Auxins, Cytokinins, Kinetin, Zeatin, 6-benzylaminopurine, Diphenylurea, Thidiazuron, Triacontanol, Karrikins, Strigolactones, and Gibberellins.

Example Fractionated Nonviable Microbes include heamatococcus algae, spirulina, chlorella, yeast such as saccharomyces cerevisiae, fungi, bacteria, archaea, and viruses including bacteriophages, archeophages, and plant viruses.

### Processing the MEBA

According to one embodiment of the invention the method of creating the MEBA comprises the step of preparing the Compound Matrix and the Carrier System simultaneously. The Carrier System and Compound Matrix are prepared and mixed under anaerobic or aerobic conditions and should be milled to between 0.1 and 1000 μm. In another embodiment the Carrier System and Compound Matrix are milled to between 100 and 800 μm. In the best mode embodiment the Carrier System and Compound Matrix are milled to between 300 and 500 μm sized particles. The finished MEBA pellets should have ultra-micropore structures of 0.1-30 μm.

The Carrier System and the Compound Matrix are prepared and mixed under anaerobic or aerobic conditions, depending on the need to influence the Eₕ in the target soil or gastrointestinal system of animals and humans. In general, the Eₕ should be between −500 mV and +900 mV. For a reducing environment the Eₕ should be between −500 mV and −100 mV. In most cases, it is preferred to create an anaerobic low Eₕ environment, below −100 mV, for the promotion of beneficial anaerobic bacteria.

According to one embodiment of the invention the method of creating the MEBA comprises the steps of preparing the Carrier System and then absorbing the Compound Matrix. The Carrier System is prepared under anaerobic conditions and should be milled to between 0.1 and 1000 μm. Another embodiment mills the Carrier System to between 100 and 800 μm. The best mode embodiment mills the Carrier System to between 300 and 500 μm size particles.

The Compound Matrix is then absorbed by the Carrier System under the desired Eₕ conditions. For strict anaerobic and low Eₕ conditions, the Eₕ should be between −500 mV and 0 mV. The best mode embodiment Eₕ for anaerobic conditions are between −400 mV and −100 mV. A low Eₕ, providing anaerobic conditions, is preferred for the promotion of beneficial bacteria. Once combined, the MEBA is formed.

The MEBA may be formed prior to administration in various ways: Freshly prepared in granules, Frozen preparation to be thawed prior to administration, Dried or freeze dried, Capsules, Tablets, Liquids, Paste. The dosage should be adjusted to an amount of active material corresponding to the need.

### Supernatants

Some embodiments also include the addition of a Supernatant, prepared as described below, to the MEBA.

These cell free Supernatants are prepared from a culture of Cultivated Microbes, including those derived from single strains, or from a combination of up to five thousand keystone species. Supernatants may also be derived from a natural consortium, synthetic consortium, or a whole ecosystem comprising one or more of the following: strict anaerobic bacteria, strict anaerobic archaea, facultative anaerobic bacteria, facultative anaerobic archaea, aerobic bacteria, aerobic archaea, eukaryotic microorganisms, and viruses. Example eukaryotic microorganisms include, but are not limited to fungi, algae, and protozoans.

These cell free Supernatants contain microbiota-derived components that are present in healthy soils or various parts of the body of healthy animals and humans, including, but not limited to, the gastrointestinal tract, skin, mucosal surfaces, ear, mouth, nose, eyes, urinary tract, and reproductive organs. They may include fatty acids, proteins, peptides, quorum sensing compounds, nucleotides, nucleotide derivatives, hydrogen sulfide, ammonia, and many different volatile and non-volatile fermentation products. These Supernatants provide metabolic factors beneficial to the growth of microbiota.

Sourcing of Bacteria and Microbes for a Supernatant from Soil and Animals

In one embodiment, natural soils from different parts of the world are selected.

Preferably, they have never been exposed to artificial chemical compounds, herbicides, pesticides, antibiotics, or non-biological methods of soil treatment. Soils are considered suitable if they have been cleared by an agronomist after comprehensive examination, including but not limited to, the analysis of pH, chemical composition, type of humus, water retaining capacity, free of relevant pathogens, free of manmade pesticides, herbicides and environmental toxins, and possessing a healthy and diverse microbiota.

Animal donors are considered suitable if they have been cleared by a veterinarian after a comprehensive examination, including but not limited to, analysis of the blood, gastrointestinal contents and stools, free of relevant pathogens, possessing a healthy and diverse microbiota, and particularly possessing a healthy and diverse gastrointestinal microbiota. Animal donors living ecologically integrated in their original natural habitat are preferred. The microbes for cultivation to prepare the supernatant may be sourced from any body part, including but not limited to, the gastrointestinal tract, skin, mucosal surfaces, ear, mouth, nose, eyes, urinary tract, and reproductive organs of healthy animals.

Fresh feces from healthy animals, including chickens, ducks, pigs, cows, horses, camels, dogs, cats, salmon, shrimp, and lobsters have all been used as sources for anaerobic cultivation of microbiota. Typical cultivation yields between 10⁹ etween¹⁰ bacteria after seven days.

Sourcing of Bacteria and Microbes for a Supernatant from Humans

In one embodiment, Supernatants derived from human microbiota are added to the MEBA. Microbiota donor candidates are asked to complete questionnaires that include biographic information, personal and family clinical history, dietary and behavioral habits, before providing blood and fecal samples for analysis.

The suitability of the donors is determined upon certain criteria. Inclusion criteria for men and women include having an average body mass index (“BMI”) between 19 and 25. Further a healthy diet and a healthy lifestyle is essential. Healthy dietary habits may be defined by the United States Food and Drug Administration (“FDA”), the European Food Safety Authority (“EFSA”), or another authority. Candidates are asked to complete a Food Frequency Questionnaire (“FFQ”), which are used to calculate their nutritional intake with reference to different guidelines.

The stool samples are analyzed to establish the composition of the microbiome. This is done by using metagenomic techniques. The outcome from the analysis of the donor stool is compared to reference samples of pathological samples with low biodiversity such as patients with Recurrent Clostridium difficile infection. If the biodiversity in the donors is greater that those with pathological microbiota patterns, and/or have positive microbial markers such as Faecalibacterium prausnitzii or Akkermansia muciniphilia, they have a high degree of suitability.

Exclusion criteria: personal or family history of gastrointestinal diseases, psychiatric disorders, neurological disorders, behavioral disorders (such as autism, alcoholism), metabolic disorders, immune or autoimmune disorders, chronic infections. Excluded are also persons with a recent exposure to communicable disease, (e.g., by risky behaviors such as tattoo, unsafe injections, piercings) or recent travel to risky countries.

Also use of any of the following medications within the last ninety days: antibiotics, antidepressants, immune modulating drugs or proton pump inhibitors are criteria for exclusion.

Blood and stools are tested for a number of diseases here listed in non limiting examples: HIV, HSV, HPLV-I, HPLV-II, all types of viral Hepatitis, Treponema pallidum, EBV Epstein-Barr virus, CMV Cytomegalovirus, enteric pathogens, nematodes and parasitic protozoan.

After the donor's laboratory screening is found to be suitable, they will complete a form again before donating the stool sample, where they are controlled for any acute gastrointestinal infection or recent used of medication.

Human microbiota may be collected from any body part including but not limited to the gastrointestinal tract, skin, mucosal surfaces, ear, mouth, nose, eyes, urinary tract and reproductive organs of healthy humans.

According to one embodiment human microbiota may be collected from any part of the gastrointestinal system including but not limited to the oral cavity, gums, teeth, tongue mucosal surfaces, naso-pharynx, esophagus, stomach, duodenum, bile and pancreatic ducts, jejunum, ileum, cecum, colon, stool samples, the luminal contents of any part of the gastrointestinal system, in particular the large intestine, or from a mucosa biopsy. Preferred sources are the cecum, colon ascendens, colon transversus, colon descendens, and sigmoid colon.

According to one embodiment a pool of feces from two or more human individuals previously pre-screed according to established guidelines, both less than thirty years of age and having an average BMI of 22, living on a balanced western diet was collected and used for preparing the bacteria free supernatant.

### Cultivation of Bacteria and Microbes for a Supernatant

In one embodiment, microbiota are anaerobically cultivated in a single-vessel continuous batch. The microbiota sample may derive from soil or any animal species, including humans. The microbiota may be obtained from a pool of sources or a single source. In one embodiment, the microbiota sample is from a single healthy donor, selected from a plurality of donors, using different criteria for assessing its suitability.

The microbiota sample can be directly used for cultivation or may be treated and processed prior to cultivation. Non-limiting examples of processing include blending, filtering, sieving, freezing, or removing undesired organisms.

A person skill in the art can determine the presence or absence of the microorganisms and viruses of interest. This may be done by employing a variety of techniques such as preparing a DNA isolation, using primers to selectively amplify the 16s rRNA gene through PCR, and finally utilizing DGGE or preferably a sequencing technique, such as pyrosequencing.

Phages may remain or be removed from the Supernatant. To remove phages, one would add a quarter volume of a solution 20% PEG 8000, 2.5M NaCl to the Supernatant, incubate one hour on ice, and then spin at 10000 rpm for 15 min at 4° C.

In one embodiment, the microbiota sample is comprised of one or more bacteria.

Preferably, in another embodiment, the sample is comprised of ten or more bacteria. In one embodiment, the microbiota sample is comprised of more than one percent bacteria. A higher concentration is preferred. Another embodiment is comprised of more than twenty percent bacteria, and another embodiment is comprised of more than fifty percent bacteria. Other embodiments may be comprised of one or more species of microorganisms from archaea, bacteria, eukaryote and viruses, or any combination of the four.

It is desirable to maintain a maximum level of biodiversity from the original microbiota sample. In one embodiment, the Simpson's index of biodiversity is not lower than 0.001 of the original microbiota sample. Preferably, the biodiversity is maintained at higher levels. In other embodiments, the Simpson's biodiversity index is maintained at 0.1, 0.2, 0.3, 0.4, 0.5, 0.6, 0.7, 0.8, 0.9 or up to 0.999 of the original sample.

A first step for the microbiota cultivation comprises using a saline or other suitable buffer to mix the microbiota into a dispersion. This mixing may be done under anaerobic or aerobic conditions. Anaerobic conditions are preferred for the promotion of beneficial anaerobic bacteria. If aerobic conditions are used substances or gases may be added during the process that protect the integrity of obligate anaerobic organisms. This mixing is done under sterile conditions, but can also allow for the addition of selected beneficial microbial strains. The final concentration of the original microbiota in the dispersion is between 1% and 99%. In one embodiment, the concentration of the microbiota suspension range is between 10% and 60%. In another embodiment, the concentration is 25%. In one embodiment, the buffer used for preparing the slurry is a sodium phosphate buffer (0.1 M, pH 7.0). In another embodiment, used a physiological saline solution with a concentration of 0.9% NaCl. The dispersion can be passed through a filter before further processing or use.

The cultivation of the microbiota is conducted under an oxygen free (anaerobic) or oxygen containing (aerobic) atmosphere. In the case of cultivation in the presence of oxygen, substances may be added to allow the simultaneous co-cultivation of obligate (strict) anaerobic and facultative anaerobic, micro aerophilic and aerobic organisms. The oxygen level, pH and redox potential may be monitored during this cultivation.

In one embodiment the propagation of the microbiota is conducted under an oxygen free, anaerobic, environment. Anaerobic conditions may be created by flushing nitrogen or other inert gasses into the reaction chamber, or by other means. Anaerobic conditions are preferred. The monitoring of the pH is continuously done during the cultivation in range between 2.1 and 7.9. In one embodiment, the pH is controlled in a range between 5.0 and 7.5, preferably the pH is controlled in a range between 6.5 and 7.0. In one embodiment pH is monitored by the addition an acidic acid (e.g., HCL) or a base (e.g., NaHCO3).

In one embodiment, the growth medium is continuously pumped into the cultivation.

The growth medium may be composed of a source of peptides, vitamins, minerals, carbohydrates, lipids and other substances suitable for the cultivation of microbiota.

In one embodiment, the growth medium is composed of DMEM, 20% FBS, 1% glutamine, 1 mg.ml-1 pectin, 1 mg.ml-1 mucin, 5 μg.ml-1 Hemin and 0.5 μg.ml-1 Vitamin K1.

In one embodiment the growth medium is composed (in g/L) of starch (3), glucose (0.4), yeast extract (3), peptone (1), hen yolk (1.25), N-Acetyl glucosamine (2), N-acetyl galactosamine, and L-cysteine (0.5) in distilled water.

In one embodiment, the growth medium is composed (in g/L) of CaCl2 (0.2), MgSO (0.2), K2HPO4 (1.0), KH2PO4 (1.0), NaCl (2.0), NaHCO3 (10.0), Citric acid (0.2), Na2HPO4 (3.1), Peptone (Difco) (10), Yeast extract (10), Cysteine (HCl com (1), humic acid (2), cholesterol (3), freeze dried hen yolk (9.5) in distilled water.

In one embodiment, the growth medium is composed (in g/L) of CaCl2 (0.2), MgSO (0.2), K2HPO4 (1.0), KH2PO4 (1.0), NaCl (2.0), NaHCO3 (10.0), Citric acid (0.2), Na2HPO4 (3.1), Peptone (Difco) (10), Yeast extract (10), Cysteine (HCl.H2O) (1), ascorbic acid (1), Phycocyanin (0.2), riboflavin (0.0001), glutathione (0.01), humic acid (2), cholesterol (3), freeze dried hen yolk (9.5) in distilled water.

In one embodiment, the growth medium is composed (in g/L) of CaCl2 (0.2), MgSO (0.2), K2HPO4 (1.0), KH2PO4 (1.0), NaCl (2.0), NaHCO3 (10.0), Citric acid (0.2), Na2HPO4 (3.1), Peptone (Difco) (10), Yeast extract (10), Cysteine (HCl t, th(1), ascorbic acid (1), Phycocyanin (0.2), riboflavin (0.0001), glutathione (0.01), humic acid (1), fulvic acid (1), cholesterol (3), freeze dried hen yolk (9.5) in distilled water.

In one embodiment, the growth medium is composed (in g/L) of CaCl2 (0.2), MgSO (0.2), K2HPO4 (1.0), KH2PO4 (1.0), NaCl (2.0), NaHCO3 (10.0), Citric acid (0.2), Na2HPO4 (3.1), Peptone (Difco) (10), Yeast extract (10), Cysteine (HCl t, th(1), ascorbic acid (1), Phycocyanin (0.2), riboflavin (0.0001), glutathione (0.01), cholesterol (3), freeze dried hen yolk (9.5) in distilled water.

In one embodiment, the growth medium is composed (in g/L) of CaCl2 (0.2), MgSO (0.2), K2HPO4 (1.0), KH2PO4 (1.0), NaCl (2.0), NaHCO3 (10.0), Citric acid (0.2), Na2HPO4 (3.1), Peptone (Difco) (10), Yeast extract (10), Cysteine (HCl.H2O) (1), ascorbic acid (1), Phycocyanin (0.2), riboflavin (0.0001), glutathione, mucin (2), freeze dried hen yolk (9.5) in distilled water.

Cultivation is performed by managing numerous variables to obtain a Cultivated Microbiota during the exponential growth phase or at steady state, keeping the maximal biodiversity of the original microbiota sample. The exponential growth phase and the steady state is determined based on temporal analysis of post biotic compounds originating from the cultivated microbiota such as Short Chain Fatty Acids (“SCFA”). At any time during the exponential phase or at the steady state the biodiversity and composition of the cultured microbiota can be analyzed.

In one embodiment, a composition of bacterial community samples at any time point of the exponential growth phase or at steady state are studied using metagenomic analyses, such as shot-gut analysis and 16s. The Nucleic acids are extracted and further processed according to known techniques. Data obtained is analyzed by bioinformatics methods for taxonomic profiling using reference databases. Diversity is established using a biodiversity index (e.g., Shannon's or Simpson's) including richness and evenness parameters.

In one embodiment, the cultivation time (“CT”) is in a range between 1 hour and 144 hours. In another embodiment, CT is in a range between 12 hours and 86 hours. In another embodiment, CT is in a range between 12 hours and 76 hours. In another embodiment, CT is 24 hours.

In one embodiment, the CT is in a range between 72 hours and 30 days. In another embodiment, the CT is between 1 day and 28 days.

### Preparation of the Supernatant

A Supernatant is prepared by removing cellular and fibrous matter from a microbiota culture.

The microbiota for preparing the cell free Supernatant may be obtained from healthy natural organic soils, healthy animals, or humans.

One embodiment of the invention relates to a composition obtained after removing live bacteria and solid (e.g., fibrous) matter from a cultured microbiota comprising obligate and facultative anaerobic bacteria of at least three of the following five Phyla: Bacteroidetes, Firmicutes, Proteobacteria, Actinobacteria and Verrucomicrobia. In another embodiment, the cultured microbiota may also be comprised of archaea, preferably Methanogens, Euryarchaeota, or Crenarchaeota Phyla, Eucaryotic microbes (e.g., fungi of genera Saccharomyces, Malassezia, and Candida) and protozoans.

In one embodiment, the Supernatant is dried, or freeze dried into a powder. In another embodiment, the supernatant may be a frozen solution, fresh from cultivation. In another embodiment, the supernatant may be comprised of a cryoprotectant, a lyoprotectant, or a preservative. The end product may further be processed or formulated by reconstituting the frozen or freeze-dried powder in a sterile saline liquid.

Suitable cryoprotectants are triols (e.g., glycerol), sulfoxides (e.g., dimethyl sulfoxides), diols and derivatives (e.g., polyethylene glycol, ethylene glycol, diethylene glycol), polyalcohols (e.g., mannitols, sorbitols, dulcitols), monosaccharides (glucose, xylose), trisaccharides, and polysaccharides (e.g., dextran, dextrin), amides (e.g., acetamide, methylacetamide, dimethylfomamide, succinimide), heterocyclic compounds (e.g., methylpyrrolidone), amino acids and carbonic acids (e.g., glycine, proline, glutaric acid, ammonium acetate, EDTA), proteins and peptides (e.g., albumins, peptones), and nonionic surfactants (e.g., Tween 80, triton). In one embodiment, the concentration of these cryoprotectants is between 1% and 20% (v/v). In another embodiment, the concentration of these cryoprotectants is between 5% and 10%. In one embodiment, the cooling rate is between 0.5 and 5.0° C/min. In another embodiment, the cooling rate is 1.0° C./min to minimize cellular damage due to osmotic imbalance and ice crystal formation. In one embodiment, frozen samples are stored at −80° C. In another embodiment, frozen samples are stored at −20° C.

Preferably, the Cultivated Microbiota is free from the following microbes: hepatitis viruses A, B and C, cytomegalovirus (“CMV”), Epstein-Barr virus (“EBV”), human immunodeficiency virus (“HIV”), Calici- and Rotavirus, Salmonella, Shigella, Campylobacter, Yersinia, and protozoan cysts. Preferably it is also free from material selected from Extended spectrum- and Metallo-beta-lactamases, metabolites of pharmaceutical active substances, and xenobiotics.

As known in the art, a method that separates the cellular and fibrous components of a microbiota culture from a liquid supernatant normally comprises a centrifugation step, and/or a filtration step with a 0.22 micron filter, wherein all of the live bacteria and other microorganisms are removed. In one embodiment, the separation may be after twenty-eight days of cultivation. In another embodiment, the separation occurs between one and twenty-one days. In another embodiment, the separation occurs between six and ten days.

In one embodiment, the separation occurs at a temperature between 15 and 40° C. In another embodiment, the separation occurs at room temperature.

In one embodiment, centrifugal tubes comprising the cultivated microbiota are subjected to centrifugation for thirty minutes, at a rotation speed corresponding to a G-force of 1000. A foamy surface layer of lipid-like material is removed, the liquid interphase decanted and then re-centrifuged for another thirty minutes at 1000 G. Remaining bacteria and fibrous matter forms a sediment at the bottom of the tube. The decanted liquid is then subjected to ultrafiltration through a 0.22-micron filter to ensure sterility of the resulting Supernatant. The Supernatant is comprised of at least one of the following: fragments of bacteria, bacteriophages, metabolites of microbiota, indole compounds, quorum sensing compounds, fatty acids, amino acids (e.g., cysteine), and volatile and non-volatile fermentation products preferably in a reduced Eₕ state.

The obtained Supernatant may then be used as an addition to the MEBA.

### Additional Substances

MEBA can be used in combination with other substances or compounds for practical use, or to aid results.

In one embodiment, MEBA may further comprise pharmaceutically-acceptable excipients, including any physiologically inert, pharmacologically inactive material known to one skilled in the art. Pharmaceutically-acceptable excipients include, but are not limited to, vehicles, adjuvants, carriers, diluents, solvents, co-solvents, buffer systems, surfactants, preservatives, sweetening agents, flavoring agents, pharmaceutical grade dyes or pigments, viscosity agents, isotonizing agents, soothing agents, and antioxidants. Some embodiments further comprise pharmaceutically acceptable auxiliary substances, such as pH adjusting and buffering agents, tonicity adjusting agents, stabilizers, and wetting agents.

### Uses and Administration of MEBA

MEBA may be useful in correcting the Eₕ of any microbial environment, towards the facilitation of fostering the growth of beneficial microbiota. Primary uses of MEBA include soil, animal, and human microbial environments.

MEBA may be used for treatment of dysbiosis and prophylaxis of diseases in soils. Such uses include the treatment of fungal or bacterial diseases such as Cylindrocladium, Pythium, Phytophthora, and Rhizoctonia, Erwinia (soft rot), Rhizomonas (corky root of lettuce), and Streptomyces (potato scab, soft rot of sweet potatoes). In one embodiment, MEBA is added to the soil in a concentration of 1-100%. Another embodiment in a concentration of 5-50%. Another embodiment in a concentration of 10-20%.

An example MEBA administration to soil is as follows. A Carrier System of Pure Silica (SO2) is heated to 130° C. to eliminate microbes. Then, 52% of the heated Pure Silica is milled to a particle size between 500 and 1000 μm, and the other 48% is milled to a particle size between 20 and 100 μm. The powder is mixed under anaerobic conditions with 2% anthraquinone-2,6-disulfonic acid. A Compound Matrix of 1% chitin, milled to 2-10 μm, and a small amount of deionized water comprising 5% dissolved reduced cysteine, is mixed with the Carrier System to form a thick paste. The paste is pressed through a perforated plate, dried, and made into small pellets of 3-5 mm in diameter. The MEBA pellets have ultra-micropore structures of 0.1-30 μm, where microbes can establish themselves, and interact with the surrounding environment. The dried pellets are packed in airtight containers for soil treatment.

MEBA may be used in the treatment and prophylaxis of diseases and conditions in humans. Examples of disorders that can be treated or prevented by MEBA are diarrheas following antibiotic treatment (Antibiotic Associated Diarrheas), especially diarrheas caused by Clostridium difficile.

Other therapeutic areas are Irritable Bowel Syndrome (“IBS”), Celiac Disease and Inflammatory Bowel Disease for example Ulcerative Colitis, Crohn's Disease, Microscopic Colitis, and Pouchitis. Other treatment areas are iatrogenic disturbance/dysbiosis of the intestinal microbiota, (e.g., following radiation therapy, chemotherapy and in connection with transplantations). Examples of other diseases that can be treated or prevented with MEBA are neurological diseases, Parkinson's Disease, Alzheimer's Disease, Lou Gehrig's Disease or ALS-Amyotrophic Lateral Sclerosis, Multiple Sclerosis; behavioral/psychiatric disorders (e.g., Autism, Asperger Syndrome, Attention Deficit Hyperactivity Disorder (ADHD) and Depression) alcoholism-cravings, Sleeping disorders (e.g., caused by Restless Leg Syndrome (RLS)), Rheumatologic diseases (e.g., Rheumatoid arthritis), systemic/metabolic disorders (e.g., Hypertension, Obesity, type-1 Diabetes, and type-2 Diabetes), and neoplastic/tumor diseases of the gastrointestinal tract e.g. adenomas and colon carcinoma. The invention can also be used in the treatment of Chronic Fatigue syndrome.

MEBA may be administrated to animals and humans with an appropriate device such as a naso-duodenal tube, gastroscope, colo/sigmoidoscope, enema, or in a freeze dried galenic preparation (e.g., gastric acid resistant capsule, feed pellets, nano-encapsulation, paste, drink, or suppository).

In one embodiment, MEBA is applied to the gastrointestinal tract of animals or humans at least once daily during the course of a number of days (e.g., 1 to 365 days in a year), depending on the nature, cause, and severity of the dysbiosis.

### Examples Given to Animals and Humans

In one embodiment, 1-100 grams of freeze dried MEBA is given to a human, preferably 20-40 g, or the equivalent amount in paste or liquid suspension. The MEBA particles may be spherical, oblong, or of any shape and size. Preferably, the size if the particles are from 0.1 mm to 10 mm in diameter, and have micropore structures of 0.1-30 μm. MEBA may be administered according to the following non limiting examples: in the form of a liquid suspension through the oral or the nose cavity, through the colon with a naso-duodenal tube, gastroscope, colo/sigmoidoscope, enema, or in any other form in a suitable galenic preparation. The treatment of an animal or human with the invention may or may not be preceded by a lavage, an antibiotic treatment, and/or motility reducing substances.

In one embodiment, a Carrier System of Loess soil is dried at 130° C. to eliminate microbes. The soil is milled to a fine powder and passed through a mesh to obtain a particle size between 20 and 50 μm. Under strict anaerobic and low Eₕ conditions (Eₕ<0 mV, more preferred <400 mV), a Compound Matrix comprising 2% bodyweight glutathione and a Supernatant from a strictly anaerobically cultivated bacteria consortium of keystone bacterial species, or a whole ecosystem of microbiota, is mixed to a paste with another Microbe Free Supernatant obtained from the anaerobic cultivation of a consortium of anaerobic microbes from the mucosal lining and lumen of the cecum and/or colon of a healthy animal or human. The paste is pressed through a perforated plate and freeze dried under vacuum, breaking it up into small granules of about 1 to 3 mm in size. The granules are coated to resist stomach acid and enable a delayed release in the colon (after 6 to 8 hours of oral administration to a human).

In one embodiment, a Carrier System of Graphene Aerogel is sterilized to eliminate microbes. The Graphene aerogel particles are between 300-800 μm. Under strict anaerobic and low Eₕ conditions (Eₕ<0 mV, more preferred <400 mV), a Compound Matrix solution of 2% L-Cysteine dissolved in a supernatant from a strictly anaerobically cultivated Akkermanisa muciniphilia to form a MEBA. The MEBA is freeze dried under vacuum and filled in slow release gastric acid resistant capsules with targeted release in the colon.

In one embodiment, a Carrier System 50/50 proportion of Loess soil and Pumice is dried at 130° C. to eliminate microbes. The materials are milled to a fine powder and passed through a mesh to obtain a particle size between 20 and 50 μm. Under anaerobic conditions, the powder is mixed with a Compound Matrix of reduced physiological solution comprising 1% reduced glutathione, 0.5% L-cysteine, 0.5% phycocyanin, 0.5% ellagic acid, 1% tocopherol acetate, 0.5% riboflavin, and 0.5% Ascorbic acid, to a paste. The paste is pressed through a perforated plate and freeze dried under a vacuum, breaking it up into small granules of about 1 to 3 mm in size. The granules are coated to resist stomach acid and enable a delayed release in the colon (after 6 to 8 hours after oral administration to a human).

In one embodiment, a Carrier System of 50/50 mixture of Zeolite and Silica is dried at 130° C. to eliminate microbes. The soil mineral blend is mixed with an equal part of compounds rich in quinones, subsequently milled to a fine powder, and passed through a mesh to obtain a particle size between 20 and 50 μm. A Compound Matrix comprising 10% L-Cysteine, 5% Ascorbic acid, 3% Mucin, and 12% by weight in de-ionized water is mixed together with the Carrier System under strict anaerobic and low Eₕ conditions (Eₕ<0 mV, more preferred <400 mV), to form a paste. The paste is pressed through a perforated plate and freeze dried under vacuum, breaking it up into small MEBA granules of about 1 to 3 mm in size, having micropore structures of 0.1-30 μm. A Supernatant from a strictly anaerobically cultivated bacteria consortium of keystone bacterial species, containing Akkermansia muciniphilia and Faecalibacterium prausnitzii, is absorbed on the freeze dried MEBA granules. The granules are again freeze dried under vacuum and anaerobic conditions to retain a low redox value. The granules are coated to resist stomach acid and enable a delayed release in the colon (after 3 to 8 hours of oral administration to a human).

### Example MEBA Applications

Patients are eighteen years or older, with a confirmed recurrent Clostridium difficile infection. Patients may or may not undergo an antibiotic treatment and/or a bowel lavage prior to being treated with the invention. The MEBA granules are given orally and ingested with some water. Approximately 10-20 grams of granulate are given at one time.

In one embodiment, a thirty-six year old female suffering from years of irritable bowel syndrome, musculoskeletal pain, and Chronic Fatigue Syndrome was given the Loess and Pumice formulation described above, in a liquid suspension. At the time of treatment, she had strong abdominal pain and a score on chronic fatigue consistent with ME-diagnosis grade I. The MEBA was administrated by a gastroscope as a 20 ml liquid solution, distally in the duodenum of the patient. The patient reported her abdominal pain disappeared almost immediately after the intervention, she was pain free for three days, and had less diarrhea.

In one embodiment twenty patients suffering from Ulcerative colitis (“UC”) with mild to moderately active UC were randomized into receiving the invention according on one of the above-mentioned embodiments or a placebo, ten subjects in each group. The treatment or placebo was administered by capsules. The primary outcome was steroid-free remission of UC, defined as a total Mayo score of </=2 with an endoscopic Mayo score of 1 or less at week eight. The primary outcome was achieved in six of the ten participants who received the invention as compared to one in the placebo group.

In one embodiment, a sixty-five year old female suffering from disturbed sleep due to restless leg syndrome (RLS) was treated with the invention according to one of the embodiments above, orally as a freeze-dried powder in capsules. The patient reported her abdominal discomfort disappeared and her sleep improved gradually over ten days.

In one embodiment, ten patients suffering from Autism were randomized into receiving the invention according to one of the above-mentioned embodiments or a placebo, five subjects in each group. The treatment or placebo was administered by capsules. The primary outcome was: Change in Childhood Autism Rating Scale (“CARS”) from baseline to ten weeks. The secondary outcome measures: Change in Daily Stool Log (“DSL”) from baseline to ten weeks. The primary outcome was achieved in five of the ten participants who received the invention as compared to zero in the placebo group. The secondary outcome was achieved in eight of the ten participants who received the invention as compared to one in the placebo group.

In one embodiment, four patients suffering from depression were randomized into receiving the invention according to one of the above-mentioned embodiments or a placebo, two subjects in each group. The treatment or placebo was administered by capsules. The primary outcome was depressive symptoms as measured with the Hamilton Rating Scale for Depression. The primary outcome was achieved in two of the two participants who received the invention as compared to zero in the placebo group.

In one embodiment, six patients suffering from systemic sclerosis (“SSc”) were randomized into receiving the invention according to one of the above-mentioned embodiments or a placebo, three subjects in each group. The treatment or placebo was administered by capsules. The primary outcome was Clinical SSc-related GI parameters and the change in the UCLA GIT score. The primary outcome was achieved in two of the three participants who received the invention as compared to zero in the placebo group.

In one embodiment, twelve patients suffering from obesity (BMI>30) were randomized into receiving the invention according to one of the above-mentioned embodiments or a placebo, six subjects in each group. The treatment or placebo was administered by capsules. The primary outcome was significant reduction of weight. The primary outcome was achieved in four of the six participants who received the invention as compared to one in the placebo group.

In one embodiment, eight patients suffering from rheumatoid arthritis were randomized into receiving the invention according to one of the above-mentioned embodiments or a placebo, four subjects in each group. The treatment or placebo was administrated by capsules. The primary outcome was reduction of The American College of Rheumatology 20 (“ACR20”) response. The primary outcome was achieved in two of the four participants who received the invention as compared to one in the placebo group.

In one embodiment, six patients suffering from colon carcinoma stage 1, were randomized into receiving the invention according to one of the above-mentioned embodiments or placebo, three subjects in each group. The invention was administered by capsules. Primary outcome was anti-inflammatory function of the invention demonstrated by decreasing pro-inflammatory factors and increasing anti-inflammatory factors. The primary outcome was achieved in two of the three participants who received the invention as compared to zero in the placebo group.

In one embodiment, ten patients suffering from Celiac disease were randomized into receiving the invention according to one of the above-mentioned embodiments or a placebo, five subjects in each group. The treatment or placebo was administered by capsules. The efficacy of the invention in the treatment of dysbiosis-associated disorder was assessed by number of patients who have improvement in clinical symptoms. The primary outcome was achieved in four of the five participants who received the invention as compared to one in the placebo group.

In one embodiment, eight patients suffering from HIV with GI complications were randomized into receiving the invention according to one of the above-mentioned embodiments example or a placebo, 4 subjects in each group. The treatment or placebo was administered by capsules. The efficacy of the invention in the treatment of dysbiosis-associated disorder was assessed by number of patients who have improvement in clinical symptoms. The primary outcome was achieved in 3 of the 4 participants who received the invention as compared to 0 in the placebo group.

In one embodiment, ten patients suffering from post-infectious IBS after an episode of tourist diarrhea were randomized into receiving the invention according to one of the above-mentioned embodiments or a placebo, five subjects in each group. The treatment or placebo was administered by capsules. Four subjects in the treatment group recovered after two days of taking the capsules compared to zero in the placebo group.

In one embodiment, MEBA is used for the treatment and prophylaxis of diseases in animals. It may be administrated to any animal (e.g., cattle, pigs, horse, cats, dogs, sheep, goats, chickens, ducks, geese, fish, and shrimp). It may also be used as a growth promoter in livestock, poultry farming, and in aquaculture.

In one embodiment, the target animal patients are dogs with a microbiologically confirmed relapse of C. difficile infection, after at least one course of adequate antibiotic therapy and a positive C. difficile toxin stool test. The Loess Soil and Supernatant MEBA described above was given to the dogs that recovered quickly from the diarrhea. After one month, a 16s RNA sequencing demonstrated restored biodiversity and eubiosis.

In one embodiment, the target animal patients are pigs with a microbiologically confirmed relapse of C. difficile infection, after at least one course of adequate antibiotic therapy and a positive C. difficile toxin stool test. The Loess Soil and Supernatant from healthy pigs, MEBA described above was given to the pigs that recovered quickly from the diarrhea. After one month, a 16s RNA sequencing demonstrated restored biodiversity and eubiosis.

In one embodiment, the target animal patients are broiler chickens with a microbiologically confirmed relapse of Salmonella typhimurium infection, after at least one course of adequate antibiotic therapy and a positive S. Typhimurium stool test. A Loess soil/Zeolite/Rosin Carrier System and a Compound Matrix with healthy chicken Supernatant, MEBA described above was given to the broilers that recovered quickly from the diarrhea. After one month, a 16s RNA sequencing demonstrated restored biodiversity and eubiosis.

In one embodiment, the target animal patients are salmon fish larvae. They were given MEBA in their fodder 10% by weight. The MEBA absorbed Carrier System of pectin and a reduced supernatant from Healthy salmon microbiota. The MEBA was given to the fish larvae that recovered, were much more resistant, and had a significantly higher survival and growth rate than the fish larvae fed a placebo.

The invention claimed is:

1. A Composition comprised of an inert non-digestible substance and a redox potential regulating substance.

2. The Composition of claim 1, wherein the said inert non-digestible substance is selected from the group consisting of Minerals, Aerogels, Metallic Ions, Carbon Allotropes, Charcoal, Chitin-Glucan Complexes, Quinones, Resins, Glycosaminoglycans, Polysaccharides, Latex, Waxes, and Lipids.

3. The Composition of claim 2, wherein the said inert non-digestible substance is a Mineral selected from the group consisting of Pumice, Loess, Clay, Diatomite, Zeolite, Clinoptilolite, Bentonite, Kaolinite, Silica, Quartz, and Silt.

4. The Composition of claim 2, wherein the said inert non-digestible substance is a Metallic Ion selected from the group consisting of Fe2+, Fe3+, Mn2+, and Mn4+.

5. The Composition of claim 2, wherein the said inert non-digestible substance is a Carbon Allotrope selected from the group consisting of Graphene and Graphene Aerogel.

6. The Composition of claim 2, wherein the said inert non-digestible substance is a Charcoal selected from the group consisting of Wood Charcoal, Sugar Charcoal, and Active Carbon.

7. The Composition of claim 2, wherein the said inert non-digestible substance is a Chitin-Glucan Complex selected from the group consisting of Chitin, Glucans, and Chitin-Glucan Complexes.

8. The Composition of claim 2, wherein the said inert non-digestible substance is a Quinone selected from the group consisting of anthraquinone-2,6-disulfonic acid, and anthraquinone-2-sulfonic acid.

9. The Composition of claim 2, wherein the said inert non-digestible substance is a Resin selected from the group consisting of Rosin, Glycerol Ester, Sorbitol Ester, and Mannitol Ester.

10. The Composition of claim 2, wherein the said inert non-digestible substance is a Glycosaminoglycan selected from the group consisting of Chondroitin, Chondroitin Sulphate, and Hyaluronic Acid.

11. The Composition of claim 2, wherein the said inert non-digestible substance is a Polysaccharide selected from the group consisting of Alginate, Carrageenan, Agar, Guar Gum, Arabic Gum, Starches, and Pectin.

12. The Composition of claim 2, wherein the said inert non-digestible substance is a Wax selected from the group consisting of Bee's wax, Carnauba wax, Candelilla wax, and Spermaceti.

13. The Composition of claim 2, wherein the said inert non-digestible substance is a Lipid selected from the group consisting of Ceramide, Sphingolipids, Bile acid, Cholesterol, Chenodeoxycholic acid, Ursodeoxycholic acid, Hyodeoxycholic acid, Squalane, and Squalene.

14. The Composition of claim 1, wherein the said redox potential regulating substance is selected from the group consisting of Antioxidants, Vitamins, Substances with thiol-groups, Amino Acids, Peptides, Polyamines, Enzymes, Nucleotide derivatives, Quorum Sensing Substances, Phytohormones, and Fractionated Nonviable Microbes.

15. The Composition of claim 14, wherein the said redox potential regulating substance is an Antioxidant selected from the group consisting of Ascorbic Acid, Citric Acid, Astaxanthin, Carotene, a Reduced Metal Ion, Polyphenol, Flavonoid, Fucoxanthin, Phycocyanin, Xanthine, Catechin, Ellagic acid, Chlorophyll, and Cu-Chlorophyll.

16. The Composition of claim 14, wherein the said redox potential regulating substance is a Vitamin selected from the group consisting of Vitamin A, B2, B3, B6, B12, C, D, E and K1.

17. The Composition of claim 14, wherein the said redox potential regulating substance is a Substance with a thiol-group selected from the group consisting of Cysteine, Glutathione, Thioredoxin, Methionine, Coenzyme A, Coenzyme B, Coenzyme M, Homocysteine, Lipoic Acid, Mycothiol, Bacillithiol, γ-Glu-Cys, Trypanothione, Ergothioneine, Glutathione Amide, Mono-galactosyl Diglyceride, and Galactolipids.

18. The Composition of claim 14, wherein the said redox potential regulating substance is a Polyamine selected from the group consisting of Spermidine, Spermine, Putrescine, Cadaverine, and Agmatine.

19. The Composition of claim 14, wherein the said redox potential regulating substance is an Enzyme selected from the group consisting of Catalase, Superoxide Dismutase, and Quinone Reductase.

20. The Composition of claim 14, wherein the said redox potential regulating substance is a Nucleotide derivative selected from the group consisting of Nicotinamide Adenine Dinucleotide (NAD), Nicotinamide Adenine Dinucleotide Phosphate (NADP), and Flavin Adenine Dinucleotide (FAD).

21. The Composition of claim 14, wherein the said redox potential regulating substance is a Quorum Sensing Substance selected from the group consisting of Indolic Compounds, Indole, Indole-3 Acetic Acid, Indole-3 Butyric Acid, and Indole-3 Propionic Acid.

22. The Composition of claim 14, wherein the said redox potential regulating substance is a Phytohormone selected from the group consisting of Auxin, Cytokinin, Kinetin, Zeatin, 6-benzylaminopurine, Diphenylurea, Thidiazuron, Triacontanol, Karrikin, Strigolactone, and Gibberellin.

23. The Composition of claim 14, wherein the said redox potential regulating substance is a Fractionated Nonviable Microbe selected from the group consisting of haematococcus algae, spirulina, chlorella, yeasts, fungi, bacteria, archaea, viruses, bacteriophages, and archeophages.

24. The Composition of claim 1, further comprising a Supernatant.

25. The Composition of claim 24, wherein the Supernatant is derived from at least one species of microbe.

26. The Composition of claim 24, wherein the Supernatant is derived from multiple species of microbes.

27. The Composition of claim 24, wherein the Supernatant is derived from an ecosystem.

28. The Composition of claim 27, wherein the ecosystem is comprised of at least one of the following: anaerobic bacteria, anaerobic archaea, facultative anaerobic bacteria, facultative anaerobic archaea, aerobic bacteria, aerobic archaea, eukaryota, or viruses.

29. The Composition of claim 24, wherein the Supernatant is sourced from an animal.

30. The Composition of claim 29, wherein the animal is screened for microbiota composition, pathogens, and health history.

31. The Composition of claim 24, wherein the Supernatant is sourced from soil.

32. The Composition of claim 31, wherein the soil is screened for microbiota composition, pathogens, and pesticides.

33. The Composition of claim 24, wherein the Supernatant is sourced from a human.

34. The Composition of claim 33, wherein the human is screened for microbiota composition, pathogens, and health history.

35. The Composition of claim 24, further comprising pharmaceutically acceptable excipients.

36. The Composition of claim 1, wherein the Composition further comprises micropore structures, between 0.1 micrometers and 30 micrometers, suitable for inhabitation by microorganisms.

37. The Composition of claim 36, wherein the Composition provides a low redox potential environment relative to the Composition's surroundings, allowing microorganisms to inhabit the micropores.

38. The Composition of claim 37, wherein the Composition further comprises time delayed dissolution properties, affording the controlled release of internal microbes and microbial metabolites.

39. A Method of creating a Composition, by adding a redox potential regulating substance to an inert non-digestible substance.

40. The Method of claim 39, with the additional step of sourcing the redox potential regulating substance from the group consisting of Antioxidants, Vitamins, Substances with thiol-groups, Amino Acids, Peptides, Polyamines, Enzymes, Nucleotide derivatives, Quorum Sensing Substances, Phytohormones, and Fractionated Nonviable Microbes.

41. The Method of claim 39, with the additional step of sourcing the inert non-digestible substance from the group consisting of Minerals, Aerogels, Metallic Ions, Carbon Allotropes, Charcoal, Chitin-Glucan Complexes, Quinones, Resins, Glycosaminoglycans, Polysaccharides, Latex, Waxes, and Lipids.

42. The Method of claim 39, with the additional step of heating the inert non-digestible substance to eliminate microbes.

43. The Method of claim 39, with the additional step of drying the inert non-digestible substance.

44. The Method of claim 39, with the additional step of milling the inert non-digestible substance.

45. The Method of claim 44, with the additional step of milling the inert non-digestible substance to between 0.1 and 1000 micrometers.

46. The Method of claim 39, with the additional step of filtering the inert non-digestible substance through a sieve.

47. The Method of claim 39, with the additional step of adding a Supernatant.

48. The Method of claim 39, with the additional step of adding a pharmaceutically acceptable excipient.
